(19) United States
(12) Patent Application Publication
(10) Pub. No.: US 2003/0044393 A1
(43) Pub. Date: Mar. 6, 2003

(54) Method for increasing tumor cell immunogenicity using heat shock protein

(76) Inventor: Annie-Chen Tran, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/183,972

(22) Filed: Jun. 25, 2002

Related U.S. Application Data
(60) Provisional application No. 60/303,429, filed on Jul. 6, 2001.

(51) Int. Cl. A61K 48/00

(52) U.S. Cl. 424/93.21; 424/93.7

(57) ABSTRACT

A method for induction of endogenous heat shock protein in tumor cells using a simple heat shock treatment which provides a simple and inexpensive method for augmenting antitumor vaccine potency. The method comprises administering to a mammal tumor cells which have been subject to a heat shock condition sufficient to cause induction of endogenous heat shock protein therein. Also disclosed is a composition for enhancing tumor cell immunogenicity comprising a therapeutically effective amount of attenuated tumor cells which have been subject to a heat shock condition.

### CROSS-REFERENCE

This application claims the benefit of U.S. provisional patent application No. 60/303,429, filed Jul., 7, 2001.

### FIELD OF THE INVENTION

This invention pertains to systems and methods for tumor immunotherapy. More particularly, the invention pertains to a method for increasing tumor cell vaccine immunogenicity wherein tumor cells are subject to heat shock conditions causing induction of endogenous heat shock protein therein prior to administering the tumor cells to a patient.

### BACKGROUND OF THE INVENTION

Vaccines based on autologous tumor cells have long been of interest for promoting tumor immunogenicity. Unmodified tumor cells are poor stimulators of immunity, and cellular tumor vaccines have generally involved some form of gene transfer modification to stimulate host antitumor responses. Gene transfer of MHC, co-stimulatory, chemokine, and cytokine genes in tumor cells are known. In the case of cytokines, transgene modified tumor cells expressing IL-2, IL-4, IL-6, CD2, ICAM, GM-CSF, TNF-α, TGF-β and others have been evaluated for tumorigenicity. Vaccines based on genetically modified tumor cells expressing IL-2, IFN-γ and TNF-α have been shown to suppress tumor formation and provide a short-lived, tumor-specific systemic immunity in mice.

The potential for heat shock proteins (hsp) in cancer immunotherapy has increasingly been recognized. Transgene-directed expression of induced heat shock protein 70 (ihsp70) using the HSVtk suicide gene has been shown to stimulate antitumor immunity in tumor cell lines which otherwise exhibit poor tumor immunogenicity. The nature of such killing-induced hsp in enhancing tumor cell immunogenicity is not clear. Induction of hsp may influence the activation of DC and induce infiltration of dentritic cells (DC) into tumors. Hsp may provide antitumor specific immunity via chaperoning antigenic peptides into a subset of antigen-presenting cells (APC). Expression of hsp may also enhance immunogenicity by direct presentation of Ags to γδT cells. The introduction of a new gene into vaccine development is problematic, however, and the effective use of transgene-directed expression of hsp for cancer immunotherapies has not been realized.

Purified immunogenic compositions based on hsp-peptide complexes have been shown to elicit tumor immunity, and provide a potential route to vaccines which avoids the need for gene modification of tumor cells. However, the required harvesting, isolation and purification of hsp and antigenic peptides for the compositions has limited vaccine development. Direct hyperthermia dosage to tissues to induce hsp therein has also been evaluated, but has not led to any sustained expression of hsp in tumors or surrounding tissues.

A method for increasing tumor cell immunogenicity using hsp without requiring the introduction of a gene for expression of hsp would be highly advantageous, but has heretofore been unavailable. There is accordingly a need for a such a method. The present invention satisfies this need, as well as others, and generally overcomes the deficiencies found in the background art.

### SUMMARY OF THE INVENTION

The present invention is a method for induction of endogenous heat shock protein in a variety of tumor cells using a simple heat shock treatment. The induction of endogenous heat shock proteins in accordance with the invention provides a relatively simple and inexpensive method for augmenting antitumor vaccine potency. In general terms, the invention is a method for enhancing tumor cell immunogenicity in a subject comprising administering to the subject cells which have been subject to a heat shock condition sufficient to cause induction of endogenous heat shock protein therein. Administration of heat shocked tumor cells to patients in accordance with the invention results in the regulation, either as a stimulatory manner or a suppressive manner, of the subject individual's systemic immune response, to promote tumor immunogenicity.

More specifically, the invention provides methods for enhancing tumor cell immunogenicity in a subject which comprise inducing endogenous heat shock protein in cells by exposing the cells to a heat shock condition, and administering the heat shock-treated cells to the subject. The methods of the invention may further comprise attenuating or inactivating the cells prior to their administration to a subject to render the cells proliferation incompetent.

The cells which are heat shock-treated and administered to the subject may comprise autologous tumor cells derived from the patient, bystander cells, bystander-autologous cell mixtures, allogeneic tumor cells, or various mixtures thereof. The autologous, bystander and/or allogeneic cells may be gene modified to express one or more proteins of interest. Proteins of interest include, inter alia, various cytokines and tumor antigens.

The invention also provides compositions for enhancing tumor cell immunogenicity in a subject in need thereof which comprise a therapeutically effective amount of cells which have been subject to a heat shock condition. The cells subjected to heat shock condition may comprise autologous tumor cells derived from the subject, bystander cells, bystander-autologous cell mixtures, allogeneic tumor cells, or mixtures thereof. The cells may be gene modified to express a cytokine, a tumor antigen, or other protein of interest.

The aforementioned methods and compositions, in one aspect of the invention, provide a therapeutic technique wherein subjects with tumors are administered subcutaneous injection of attenuated tumor cells that have been exposed to a heat shock condition in order to effect suppression, regression or otherwise reversal of existing tumors. Another aspect of the invention provides a cellular tumor vaccine for tumor prevention wherein subjects vaccinated with attenuated, heat shock treated tumor cells are resistant to tumors or which otherwise reject tumors when subsequently challenged with unmodified tumor cells.

### BRIEF DESCRIPTIONS OF THE DRAWINGS

FIG. 1 is a graphical representation of survivability versus time after tumor challenge for mice challenged with live unmodified B16 cells, with survivability shown for mice vaccinated with PSB (control), mice vaccinated with B16 tumor cells modified for GM-CSF expression, and mice vaccinated with B16 tumor cells modified for GM-CSF expression and subjected to heat shock condition.

FIG. 2 is a graphical representation of percent tumor free mice versus time after tumor challenge for mice vaccinated with PBS (control), mice vaccinated with B16 tumor cells modified for GM-CSF expression, and mice vaccinated with B16 tumor cells modified for GM-CSF expression and subjected to heat shock condition.

### DETAILED DESCRIPTION OF THE INVENTION

Disclosed herein are methods and compositions for induction of endogenous heat shock protein in tumor cells using a heat shock treatment. various cytokines and tumor antigens. Before the subject methods and compositions are described, it is to be understood that this invention is not limited to particular embodiments described. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

A. Definitions

“Cytokine” or grammatical equivalents, herein is meant the general class of hormones of the cells of the immune system, both lymphokines and monokines, and others. The definition is meant to include, but is not limited to, those hormones that act locally and do not circulate in the blood, and which, when used in accord with the present invention, will result in an alteration of an individual's immune response. The cytokine can be, but is not limited to, IL-2, IL-4, IL-6, IL-7, GM-CSF, γ-IFN, TNF-alpha., CD2 or ICAM. Additionally, cytokines of other mammals with substantial homology to the human forms of IL-2, GM-CSF, TNF-α, and others, will be useful in the invention when demonstrated to exhibit similar activity on the immune system. Similarly, proteins that are substantially analogous to any particular cytokine, but have relatively minor changes of protein sequence, will also find use in the present invention. It is well known that some small alterations in protein sequence may be possible without disturbing the functional abilities of the protein molecule, and thus proteins can be made that function as cytokines in the present invention but differ slightly from current known sequences. Finally, the use of either the singular or plural form of the word “cytokine” in this application is not determinative and should not limit interpretation of the present invention and claims. In addition to the cytokines, adhesion or accessory molecules or combinations thereof, may be employed alone or in combination with the cytokines.

“Systemic immune response” means an immune response which is not localized, but affects the individual as a whole, thus allowing specific subsequent responses to the same stimulus.

“Reversal of an established tumor” means the suppression, regression, partial or complete disappearance of a pre-existing tumor. The definition is meant to include any diminution in the size, potency, growth rate, appearance or feel of a pre-existing tumor.

“Rejection” means a systemic immune response that does not allow the establishment of new tumor growth.

“Challenge” means a subsequent introduction of tumor cells to an individual. Thus, for example, a “challenge dose 5 days post vaccination” means that on the fifth day after vaccination with tumor cells subject to heat shock conditions, a dose of unmodified tumor cells was administered. “Challenge tumor” means the tumor resulting from such challenge.

“Days to sacrifice” means that period of time before mice were sacrificed. Generally, mice were sacrificed when challenge tumors reached 2-3 centimeters in longest diameter, or if severe ulceration or bleeding developed.

“Attenuated cells” or “inactivated cells” means cells inactivated by rendering them proliferation incompetent by irradiation or other treatment. Such treatment results in cells which are unable to undergo mitosis, but still retain the capability to express proteins such as cytokines. Typically a minimum dose of about 3500 rads is sufficient, although doses up to about 30,000 rads are acceptable. It is understood that irradiation is but one way to inactivate the cells, and that other methods of inactivation which result in cells incapable of cell division but that retain the ability to express cytokines are included in the present invention.

“Individual”, “patient” or “subject” means a member or members of any mammalian or non-mammalian species that may have a need for the pharmaceutical methods, compositions and treatments described herein.

“Treating” or “treatment” of a condition or disease includes: (1) preventing the disease, i.e. causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

A “therapeutically effective amount” means the amount of a composition or vaccine that, when administered to a mammal or other subject for treating a disease, is sufficient to effect such treatment for the disease. The “therapeutically effective amount” will vary depending on the composition or vaccine, the disease and its severity and the age, weight, etc., of the subject to be treated.

“Mammal” means a member or members of any mammalian species, and includes, by way of example, canines; felines; equines; bovines; ovines; rodentia, etc. and primates, particularly humans. Animal models, particularly small mammals, e.g. murine, lagomorpha, etc. may be used for experimental investigations.

“Tumor cell” and “cancer cell” are used interchangeably to mean a malignant cell generally. A tumor cell can occur in and can be obtained from a solid tumor such as a sarcoma, carcinoma, melanoma, lymphoma or glioma or a more diffuse cancer such as a leukemia. Tumor cells can be obtained from a subject having a cancer, from a donor subject having a cancer that is the same or substantially similar to the cancer in the subject to be treated or from a tumor cell repository.

“Heat shock condition” means any condition sufficient to perturb or stress cells from normal homeostasis and induce heat shock proteins therein, including hyperthermia, irradiation, electrostatic discharge or oxidative chemical treatment of sufficient duration to induce heat shock protein.

“Heat shock protein” means any protein inducible via a heat shock condition, including hsp20, hsp27, hsp 60 and hsp 90.

Treating” or “treatment” of a condition or disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

The terms “subject” and “patient” mean a member or members of any mammalian or non-mammalian species that may have a need for the methods, compositions and treatments described herein.

B. General Methods for Increasing Tumor Immunogenicity Using Heat Shock Proteins

The invention provides therapeutic and vaccine methods and compositions using induction of endogenous heat shock protein in a variety of tumor cells via a simple heat shock treatment. The induction of endogenous heat shock proteins by subjecting cells to heat shock conditions in accordance with the invention provides a simple and inexpensive method for augmenting antitumor vaccine potency without the need for genetic modification of the cells for expression of heat shock protein. In a therapeutic method, tumor cells are subject to heat shock conditions, attenuated, and administered to subjects exhibiting tumors to provide high survivability in the subjects. In a vaccine or tumor prevention method, tumor cells are subject to heat shock condition and are administered to tumor free subjects. The subjects are subsequently challenged with tumor cells and exhibit high survivability.

The tumor cells used in the methods and compositions of the invention may comprise autologous tumor cells from the subject and/or allogeneic tumor cells from a commercial cell line or other source. In certain embodiments of the invention, the cells administered to subjects may, in addition to autologous and/or allogeneic tumor cells, comprise bystander cells that are engineered to express a cytokine, a tumor antigen, or other protein of interest which may regulate a subject's systemic immune response to promote tumor immunogenicity. The bystander cells may be subject to heat shock condition as well as the tumor cells. In other embodiments of the invention the tumor cells may be gene modified to express a cytokine, a tumor antigen, and/or other protein of interest.

The heat shock protein induced in accordance with the invention may comprise induced heat shock protein 70 (ihsp70). The induced heat shock protein may alternatively comprise any other endogenous heat shock protein whose expression is induced by exposure to heat shock condition and. which is capable of promoting an antitumor immune response in accordance with the invention.

The heat shock condition used to induce heat shock proteins in tumor cells may comprise a hyperthermia condition of sufficient duration and temperature to induce heat shock protein. The heat shock condition may additionally or alternatively comprise irradiation, electrostatic discharge, oxidative chemical treatment, or any other condition sufficient to perturb or stress cells from normal homeostasis and induce heat shock proteins therein.

The tumor cells exposed to heat shock conditions may be associated with a variety of different cancer types. Typically the cancer is a tumor-forming cancer, with such tumors including, by way of example and not of limitation, tumors of neuroectodermal derivation, carcinomas, tumors of mesodermal origin, or any other tumor types.

Where tumor cells are modified to express cytokines, growth factors, growth hormones, colony stimulation factors, tumor antigens, or other proteins of interest, the modified cells are irradiated or rendered proliferation incompetent and subject to heat shock conditions prior to administration to an individual. Cells modified for expression of, for example, cytokines such as interleukins 1-11; growth factors such as EGF, FGF, PDGF, and TGF somatotropins; growth hormones; or other hormones, such as FSH, LH, etc.; and colony stimulating factors, such as G-, M-, and GM-CSF may be used with the invention. These irradiated, modified cells are subject to heat shock conditions and administered in therapeutically effective amounts.

Selected embodiments of the invention utilize tumor cells expressing Granulocyte Macrophage Colony Stimulating Factor (GM-CSF). In a therapeutic method example described below, melanoma cells modified to express GM-CSF are subject to heat shock conditions and are administered to subjects exhibiting tumors to provide high survivability in the subjects. In a vaccine example, melanoma cells are modified to express GM-CSF and subject to heat shock condition are administered to tumor free subjects. The subjects are subsequently challenged with tumor cells and exhibit high survivability.

The cells subjected or exposed to heat shock conditions in accordance with the invention may be administered to cancer patients. The patients to be treated by the methods of the invention are cancer patients. The claimed methods are effective against a range of different cancer types. Typically the cancer is a tumor-forming cancer. For example, many solid tumors are amenable to treatment using the claimed invention. These tumors include but are not limited to tumors of neuroectodermal derivation (e.g., glioma), carcinomas (e.g., colon cancer, ovarian cancer), and tumors of mesodermal origin (e.g., sarcomas).

In order to assess how well the methods of the invention may be expected to work, the clinician can pre-test the efficacy of the treatment of a particular tumor type either in vitro or in vivo. For in vitro tests, cells derived from the tumor are grown in tissue culture using a variety of techniques that are well known in the art. The method described in Fick et al., U.S. Pat. No. 6,149,904 is used to assess the efficacy of using a particular engineered cell line to transfer therapeutic molecules to the tumor-derived cells. The tumor growth inhibiting effect of the invention may be assessed using a number of commonly used assays, such as cell counts or radioactive thymidine incorporation.

Administration of heat shocked tumor cells to a cancer patient can be achieved in various ways known to skilled practitioners. The cells can be injected intratumorly: the tumor, the placement of the needle and release of the contents of the syringe may be visualized either by direct observation (for easily accessible tumors such as surface tumors or tumors easily exposed by surgical techniques), by endoscopic visualization, or by electromagnetic imaging techniques such as ultrasound, magnetic resonance imaging (MRI), CT scans. The cells can also be administered via injection into the bloodstream using a cannula or catheter; the vein or artery is selected to maximize delivery of cells to the tumor or affected tissue. The cells can be injected into cerebro-spinal fluid (i.e., into intracisternal, intraventricular, intrathecal or subarachnoid compartments). In cystic or vesicular tumors or tissues, the cells may be delivered intracystically or intravesicularly.

It is contemplated that, in such cancer treatment, heat shocked tumor cells will be administered under the guidance of a physician. The concentration and number of cells to be administered at a given time and to a given patient may vary from, for example, about 10⁴ to about 10¹⁰ cells per patient. Generally, the number of cells to be administered is the amount necessary to reduce cancer cell growth and/or to destroy cancer cells and/or to eradicate the cancer in the patient. The exact number is a function of the size and compactness (or diffuseness) of the particular transformed cell mass to be treated, and the distance or accessibility of the tissue to be treated from the point of administration of the cells. More than one administration may be necessary. As with any medical treatment, the supervising physician will monitor the progress of the treatment, and will determine whether a given administration is successful and sufficient, or whether subsequent administrations are needed.

The injected cells and cells junctionally coupled thereto may be destroyed by administration of a pro-drug to the patient that will target for destruction the injected cells and cells that are junctionally coupled to them. For example, the enzyme cytosine deaminase converts 5-fluorocytosine (“5FC”) into the lethal metabolite 5-fluorouracil (“5FU”). Cells that express cytosine deaminase, when exposed to 5FC, will die as a result of the formation of 5FU. Cells that do not express the deaminase but that are junctionally coupled to deaminase-expressing cells will also die. Another gene that confers drug sensitivity upon a host cell is the thymidine kinase gene, which confers sensitivity to GCV. Administration of the pro-drug may be local or systemic and may be achieved by any of the methods for administration of the cells.

Tumor regression and other parameters of successful treatment may be assessed by methods known to persons of skill in the art. Such methods include, for example, any imaging techniques that are capable of visualizing cancerous tissues (e.g., MRI), biopsies, methods for assessing metabolites produced by the cancer tissue or affected tissue in question, the subjective well-being of the patient.

### C. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Induction of Endogenous Heat Shock Protein in HelaS₃ Human Cervical Carcinoma Cells.

HelaS₃ cells used were commercially obtained from Cell Applications, Inc of San Diego, Calif. Cultures of HelaS₃ cells (1-2×10⁶ cells/mL) were harvested (trypsinization) and subject to a heat shock condition of approximately 44° C. for approximately 2 hours by immersion in a constant temperature water bath. Cells remained healthy after subjection to heat shock in this manner. The heat shocked cells were fixed (paraformaldehyde), permeabilized (Triton X-100/BSA in TBS) and stained with FITC-conjugated mouse anti-h ihsp70. The expression level of induced ihsp70 in the heat shocked cells was evaluated with fluorescence activated cell sorting (FACS) flow cytometry analysis against an isotype-matched control. Flow cytometry analysis for expression of ihsp70 was also performed for log phase HelaS₃ cells in culture for more than 48 hours, and harvested HelaS₃ cells plated back approximately one day prior to staining.

The detected fluorescence for the isotype matched control and log phase HelaS₃ cells are shown in Table 1A and Table 1B respectively.

TABLE 1A

| | | | | X Geo. | | Y Geo. |
|---|---|---|---|---|---|---|
| Quadrant | % Gated | % Total | X Mean | Mean | Y Mean | Mean |
| UL | 0.35 | 0.23 | 2.58 | 2.49 | 78.5 | 76.61 |
| UR | 0.00 | 0.00 | — | — | — | |
| LL | 99.65 | 63.84 | 2.12 | 2.06 | 28.59 | 26.67 |
| LR | 0.00 | 0.00 | — | — | — | — |

TABLE 1B

| | | | | X Geo. | | Y Geo. |
|---|---|---|---|---|---|---|
| Quadrant | % Gated | % Total | X Mean | Mean | Y Mean | Mean |
| UL | 38.87 | 24.67 | 1.99 | 1.94 | 85.00 | 82.35 |
| UR | 0.00 | 0.00 | — | — | — | — |
| LL | 61.13 | 38.80 | 1.89 | 1.84 | 43.10 | 39.91 |
| LR | 0.00 | 0.00 | — | — | — | — |

As can be seen, the level of ihsp70 is nominally zero for the isotype-matched control in Table 1A, while ihsp70 is detectable in the log phase cells in Table 1B.

Detected fluorescence for the isotype matched control and harvested cells are shown in Table 2A and Table 2B.

TABLE 2A

| | | | | X Geo. | | Y Geo. |
|---|---|---|---|---|---|---|
| Quadrant | % Gated | % Total | X Mean | Mean | Y Mean | Mean |
| UL | 0.09 | 0.07 | 4.00 | 3.98 | 67.81 | 67.56 |
| UR | 0.00 | 0.00 | — | — | — | — |
| LL | 99.91 | 87.37 | 2.69 | 2.61 | 30.63 | 29.44 |
| LR | 0.00 | 0.00 | — | — | — | — |

TABLE 2B

| | | | | X Geo. | | Y Geo. |
|---|---|---|---|---|---|---|
| Quadrant | % Gated | % Total | X Mean | Mean | Y Mean | Mean |
| UL | 60.06 | 51.49 | 2.76 | 2.68 | 88.09 | 85.41 |
| UR | 0.00 | 0.00 | — | — | — | — |
| LL | 39.94 | 34.25 | 2.54 | 2.46 | 47.24 | 45.17 |
| LR | 0.00 | 0.00 | — | — | — | — |

The harvested HelaS₃ cells, in Table 2B, show an increase in the level of ihsp 70 over log phase cells as seen in Table 1B.

Table 3A and Table 3B show detected fluorescence for the isotype matched control and HelaS₃ cells which were subject to a heat shock condition as described above.

TABLE 3A

| | | | | X Geo. | | Y Geo. |
|---|---|---|---|---|---|---|
| Quadrant | % Gated | % Total | X Mean | Mean | Y Mean | Mean |
| UL | 0.34 | 0.26 | 3.57 | 3.41 | 78.18 | 76.61 |
| UR | 0.00 | 0.00 | — | — | — | — |
| LL | 99.66 | 76.17 | 3.38 | 3.26 | 31.12 | 29.90 |
| LR | 0.00 | 0.00 | — | — | — | — |

TABLE 3B

| | | | | X Geo. | | Y Geo. |
|---|---|---|---|---|---|---|
| Quadrant | % Gated | % Total | X Mean | Mean | Y Mean | Mean |
| UL | 94.26 | 71.11 | 3.64 | 3.53 | 176.44 | 160.71 |
| UR | 0.00 | 0.00 | — | — | — | — |
| LL | 5.74 | 4.33 | 3.59 | 3.47 | 40.35 | 35.92 |
| LR | 0.00 | 0.00 | — | — | — | — |

As can be seen from Table 3B, the heat shocked HelaS₃ cells show a substantial increase in the level of ihsp 70 from the harvested and log phase cells (Table 2B and Table 1B)

The fluorescence data of Tables 1-3 above are summarized in Table 4 below.

TABLE 4

| HelaS₃ Cells | % Total | Geometric Mean |
|---|---|---|
| Log-Phase | 39 | 5 |
| Harvested | 64 | 18 |
| Heat-Shocked | 94 | 84 |

Inducible hsp70 is present in basal levels in HelaS₃ cells during log phase, and increases approximately 16.8 fold (geometric mean) after heat shock treatment at 44° C. for 2 hours. The harvesting HelaS₃ cells and plating back to fresh media slightly increases ihsp expression about 3.6 fold (geometric mean) due to stress associated with trypsinization and harvesting.

### Example 2

Induction of Endogenous Heat Shock Protein in PC3 and LNCaP Prostate Cancer Cells.

PC3 cells and LNCaP cells were commercially obtained from BRFF of Ijamville, Md. PC3 prostate cancer cells and LNCaP prostate cancer cells were separately cultured and harvested in the manner described above for Example 1. The PC3 and LNCaP cells and were subject to heat shock treatment at 44° C. for 1 hour via immersion in a constant temperature water bath. Flow cytometry analysis results for harvested and replated cells, irradiated cells (5000 rads) and heat shocked cells (44° C. for 1 hour) are shown in Table 5.

TABLE 5

| | PC3/gm | PC3/gm | PC3/gm | LNCaP/gm | LNCaP/gm | LNCaP/gm |
|---|---|---|---|---|---|---|
| Cell | Harvested | Irradiated | heat shock | Harvested | Irradiated | Heat shock |
| % | 13 | 0 | 87 | 17 | 4 | 36 |
| Total | | | | | | |
| Geo. | 5 | 5 | 69 | 5 | 8 | 7 |
| Mean | | | | | | |

Constitutive hsp70 expression for PC3 and LNCaP cells were approximately 95% to 99% positive for all conditions. Inducible hsp70 increased approximately 14 fold (geometric mean) in heat shocked PC3 cells. In the case of the above prostate cells a heat shock condition of about 44° C. for 1 hour provided healthy cells with increased ihsp70 expression. Exposure of cells to this temperature for longer durations was non-optimal.

### Example 3

Induction of Endogenous Heat Shock Protein in B16 Melanoma Cells

B16 melanoma cells were prepared according to Vile et al., Cancer Res. 53:962 (1993). B16 melanoma cells were cultured and harvested in the manner described above for Example 1 and were subject to heat shock treatment at 44° C. for one half hour (30 minutes) by immersion in a constant temperature water bath. Flow cytometry analysis results for harvested and replated cells and heat shocked cells are shown in Table 6.

TABLE 6

| | B16gm | B16/gm |
|---|---|---|
| Cell | Harvested | Heat shocked |
| % Total | 7.06 | 83.56 |
| Geo. Mean | 29.88 | 90.43 |

Heat shock treatment in this case provided an approximately 3-fold increase in ihsp70 expression in heat shocked cells over harvested cells, with greater than 90% ihsp70 expression in the heat shocked cells.

### Example 4

Production of B16 Melanoma Cells Containing GM-CSF Encoding Sequence

The production of B16 melanoma cells containing cytokine encoding sequences may be carried out using a variety of retroviral vectors. Gene modification of B16 melanoma cells for expression of GM-CSF using the MFG vector was carried out as described by Dranoff et al. in U.S. Pat. No. 5,637,483. Briefly, vector constructs are introduced by standard methods into the packaging cell lines known as Psi CRIP and Psi CRE. CRIP packaging lines with amphotropic host range are generated by both transfection and electroporation with only small differences in efficiency. Calcium phosphate DNA coprecipitations are performed using 20 g of vector and 1 g of pSV2NEO. Electroporations are performed after linearizing 40 g of vector and 8 g of pSV2NEO using the Gene Pulser electroporator (Bio-Rad). Conditions were 190 V and 960 F. Producers were placed into selection in G418 (GIBCO) at 1 mg/ml 36 hours after introduction of DNA. Both clones and populations of producers were generated.

Viral titres were determined by Southern blot analysis following infection of B16 cells in medium containing 8 g polybrene per ml. Ten g of infected target cell DNA as well as control DNA spiked with appropriate copy number standards were digested with NheI (an LTR cutter), resolved by electrophoresis in 1% agarose gels, and analyzed by the method of Southern using standard procedures. Blots were probed with the appropriate sequences which had been labeled to high specific activity with [³²P]dCTP by the random primer method. Probes used were all full-length cDNAs. To determine if the MFG vector construct influenced the growth of B16 cells in vitro or in vivo, cells were exposed to viral supernatants and the transduced cells were characterized for their efficiency of infection and secretion of gene product.

### Example 5

Induction of Endogenous Heat Shock Protein in B16 Melanoma Cells Containing GM-CSF Encoding Sequence

The gene modified B16 cells of Example 4 were cultured and harvested as in Example 1, and were subject to heat shock treatment at 43° C. for one half hour (30 minutes) by immersion in a constant temperature water bath. Flow cytometry analysis showed greater than 80% ihsp70 expression.

### Example 6

Vaccinations

Tumor cells were trypsinized, washed once in medium containing serum, and then twice in Hanks Balanced Saline Solution (GIBCO) prior to injection. Vaccinations were administered subcutaneously in the abdomen and tumor challenges were injected in the dorsal midline of the back after anesthetizing the mice with Metaphane (Pitman-Moore). Mice were examined at 2-3 day intervals and the time to development of palpable tumor recorded. Animals were sacrificed when tumors reached 2-3 cm in diameter or if severe ulceration or bleeding developed. Animals used were 6-8 week syngeneic C57BL/6J females. When irradiated vaccines were employed, tumor cells (after suspension in HBSS) received 5000 rads from a Cesium-137 source discharging 124 rads/min. Heat shocked cell vaccines were irradiated and then heat shocked at 43° C. for 30 minutes, and stored under liquid N₂ until use.

### Example 7

Therapeutic Model Using B16 Modified to Express GM-CSF

In this example mice were challenged by injection of approximately 5×10⁴ live, unmodified B16 cells. Three days after tumor challenge, individual mice in a first group were vaccinated with approximately 10⁶ B16 cells modified for GM-CSF expression which had been heat shocked at 43° C. for 30 minutes as described above. In a second group of challenged mice individuals were vaccinated with approximately 10⁶ B16 cells modified for GM-CSF expression which were not heat shocked treated. In a third such group of challenged mice, individuals were injected with phosphate buffered saline (PBS) to provide a control group.

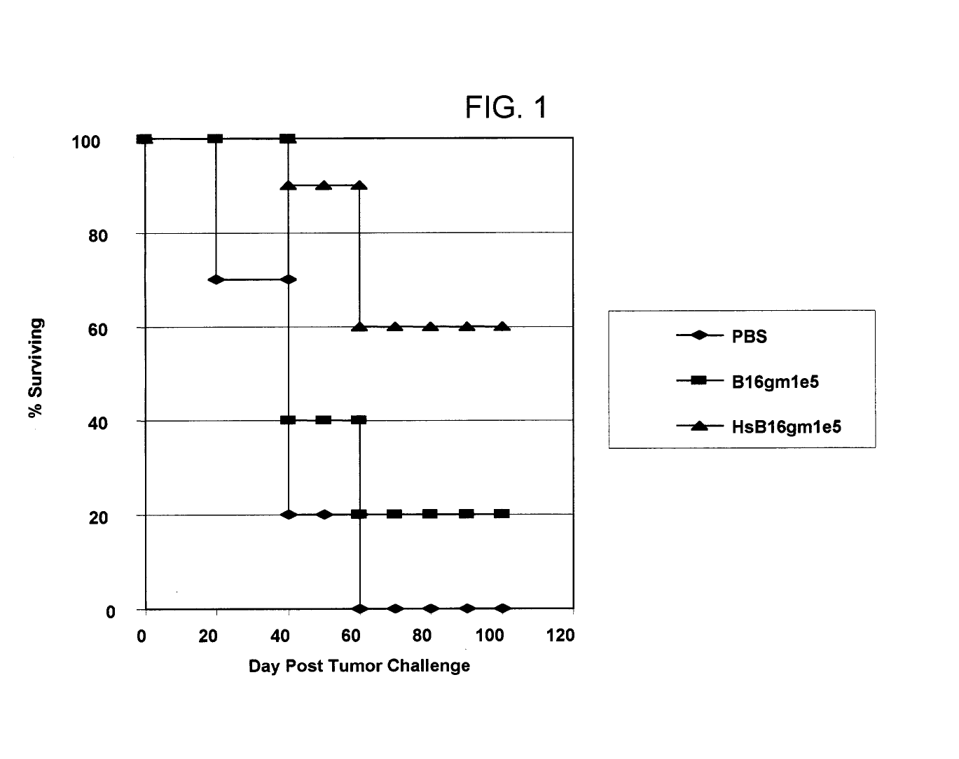

The survivability of the three groups of challenged mice is shown graphically in FIG. 1 as percent survivability versus days post tumor challenge, with the control group (PBS) shown as diamonds, the group vaccinated with GM-CSF-expressing B16 cells shown as rectangles, and with the group vaccinated with heat shocked GM-CSF-expressing B16 cells shown by triangles. As can be seen, the survival rate was zero (100% mortality) at 60 days post challenge for the control group. The mice vaccinated with B16 cells modified for GM-CSF expression but which were not heat shocked treated showed a survival rate of 20% at 60 days after challenge, and subsequently remained unchanged to 100 days post challenge. Mice vaccinated with heat,shocked B16 cells modified for GM-CSF expression showed a 60% survival at 60 days after challenge, and this survival rate remained unchanged at 100 days after challenge.

### Example 8

Vaccine Model Using B16 Modified to Express GM-CSF

In a first group of mice, individuals were vaccinated with approximately 10⁶ heat shocked (43° C. for 30 minutes) B16 cells modified for GM-CSF expression. Individuals in a second group of mice were vaccinated with approximately 10⁶ heat B16 cells modified for GM-CSF expression which were not subject to heat shock conditions. In a third, control group, individual mice were injected with PBS. Seven days after vaccination, individual mice in all three groups were challenged by injection of approximately 5×10⁴ live, unmodified B16 cells.

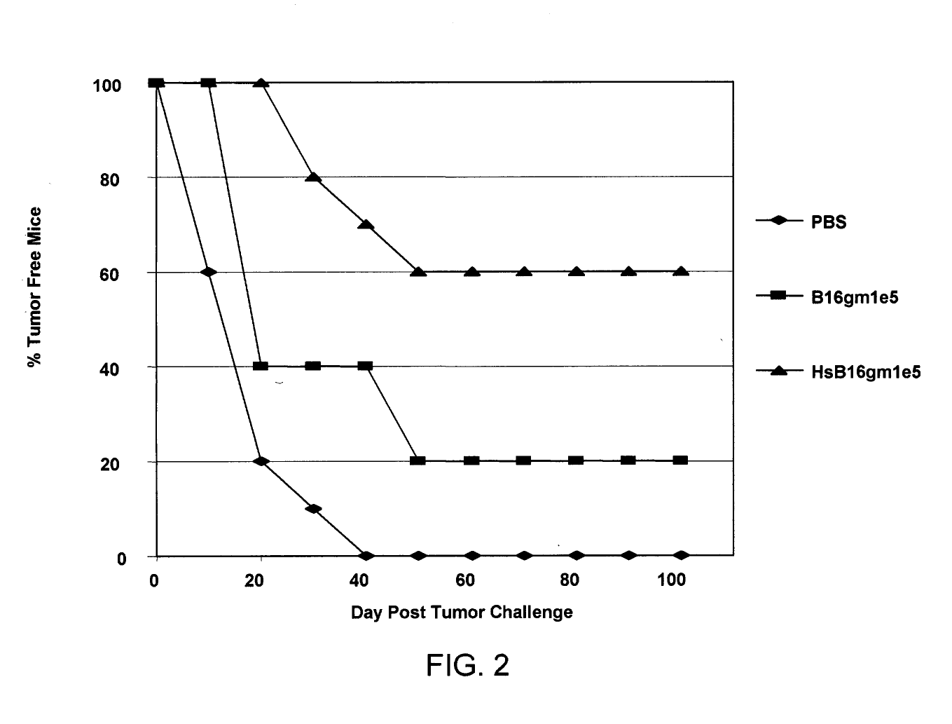

FIG. 2 graphically illustrates the percentage of tumor-free mice in each group versus days after tumor challenge. Mice in the group vaccinated with heat shocked B16 cells modified for GM-CSF expression were 60% tumor free at 50 days after challenge and remained 60% tumor free at 100 days after challenge. Mice vaccinated with GM-CSF expressing B16 that were not heat shocked were 20% tumor free at 50 days after challenge and remained at 20% tumor free at 100 days after challenge. Of the mice in the control group, 100% exhibited tumors by 40 days after challenge.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of treating a tumor in a subject comprising administering to said subject a therapeutically effective amount of tumor cells which have been subject to a heat shock condition sufficient to cause induction of endogenous heat shock protein therein.

2. The method of claim 1, further comprising transducing said tumor cells to express a protein of interest.

3. The method of claim 2, wherein said protein of interest comprises a cytokine

4. The method of claim 3, wherein said cytokine comprises GM-CSF.

5. The method of claim 2, wherein said protein of interest comprises a tumor antigen.

6. The method of claim 1, wherein said subject is a mammal.

7. The method of claim 6, wherein said mammal is a human.

8. The method of claim 1, wherein said heat shock protein comprises ihsp70.

9. The method of claim 1, wherein said heat shock condition comprises an application of about forty three degrees centigrade to said tumor cells for a duration of about thirty minutes.

10. The method of claim 1, wherein said tumor cells are autologous.

11. The method of claim 1, wherein said tumor cells are allogeneic.

12. The method of claim 1, further comprising administering to said mammal bystander cells engineered to express a protein of interest.

13. A method for enhancing tumor cell immunogenicity comprising:
(a) removing a tumor cell from a mammal;
(b) subjecting said tumor cell to a heat shock condition and causing induction of endogenous heat shock protein in said tumor cell; and
(c) administering said tumor cell to said mammal.

14. The method of claim 13, further comprising transducing said tumor cell to express a protein of interest.

15. The method of claim 14, wherein said protein of interest comprises a cytokine

16. The method of claim 15, wherein said cytokine comprises GM-CSF.

17. The method of claim 14, wherein said protein of interest comprises a tumor antigen.

18. The method of claim 13, wherein said heat shock protein is ihsp70.

19. The method of claim 13, wherein said heat shock condition comprises an application of about forty three degrees centigrade to said tumor cells for a duration of about thirty minutes.

20. The method of claim 13, further comprising administering to said mammal bystander cells engineered to express a protein of interest.

21. A composition for enhancing tumor cell immunogenicity in a subject in need thereof, comprising a therapeutically effective amount of tumor cells which have been subject to a heat shock condition.

22. The composition of claim 21, wherein said tumor cells have been transduced to express a protein of interest.

23. The composition of claim 22, wherein said protein of interest comprises a tumor antigen.

24. The composition of claim 22, wherein said protein of interest comprises a cytokine

25. The composition of claim 24, wherein said cytokine comprises GM-CSF.

26. The composition of claim 21, further comprising bystander cells engineered to express a protein of interest.
